(19) United States
(12) Patent Application Publication
(10) Pub. No.: US 2026/0335022 A1
(43) Pub. Date: Oct. 29, 2026

(54) SURGICAL PERFORMANCE ASSESSMENT

(71) Applicant: ALCON INC., Fribourg (CH)

(72) Inventors: Zhuoran WU, Fort Worth, TX (US); Nikhil SCARIA, Fort Worth, TX (US); Vignesh SURESH, Fort Worth, TX (US); Sinchan BHATTACHARYA, Fort Worth, TX (US); Aakash NAGARAJAN, Fort Worth, TX (US); Ramesh SARANGAPANI, Fort Worth, TX (US); Joseph WEATHERBEE, Fort Worth, TX (US); Lu YIN, Fort Worth, TX (US)

(21) Appl. No.: 19/650,259

(22) Filed: Apr. 16, 2026

Related U.S. Application Data
(60) Provisional application No. 63/794,923, filed on Apr. 25, 2025.

(51) Int. Cl. A61B 34/20; G06T 7/00; G06V 10/62; G06V 10/82; G06V 20/40; G06V 20/52; G06V 20/70; G06V 40/20; G16H 40/40

(52) U.S. Cl. CPC A61B 34/20; G06T 7/0012; G06V 10/62; G06V 20/44; G06V 20/52; G06V 20/70; G06V 40/28; G16H 40/40; A61B 2034/2055; A61B 2034/2065; G06T 2207/10016; G06T 2207/10056; G06T 2207/20081; G06T 2207/20084; G06T 2207/30041; G06V 10/82; G06V 20/49; G06V 2201/034

(57) ABSTRACT

A method of assessing a surgical procedure from a video may include obtaining video data capturing a surgical procedure and processing the video data to identify surgical phases within the surgical procedure. The method may also include processing at least a portion of the video data to detect one or more surgical instruments used during the surgical procedure and calculating one or more performance metrics based on the surgical phases and the detected one or more surgical instruments. The method may further include comparing the one or more performance metrics to one or more benchmark values to generate a comparative assessment and generating a performance report based on the one or more performance metrics.

### CROSS-REFERENCE TO RELATED APPLICATION

This application claims priority to U.S. Provisional Application No. 63/794,923, filed on Apr. 25, 2025, which is incorporated herein in its entirety.

### FIELD

The embodiments discussed herein relate to assessment of surgical performance.

### BACKGROUND

In many situations, surgical video recording systems have been integrated into modern operating rooms, particularly in ophthalmic surgical environments where microscope-mounted cameras capture high-resolution footage of procedures such as cataract surgery, vitreoretinal surgery, and corneal transplantation. The surgical video recording systems may store video data in digital archives maintained by hospitals and surgical centers. Most surgical video management systems organize recorded procedures using basic metadata. As a result, retrieval of specific videos from these archives relies on manual searching through metadata fields or chronological browsing of stored files.

The subject matter claimed herein is not limited to embodiments that solve any disadvantages or that operate only in environments such as those described above. Rather, this background is only provided to illustrate one example technology area where some embodiments described herein may be practiced.

### SUMMARY

According to an aspect of at least one embodiment, a method of assessing a surgical procedure from a video may include obtaining video data capturing a surgical procedure and processing the video data to identify surgical phases within the surgical procedure. The method may also include processing at least a portion of the video data to detect one or more surgical instruments used during the surgical procedure and calculating one or more performance metrics based on the surgical phases and the detected one or more surgical instruments. The method may further include comparing the one or more performance metrics to one or more benchmark values to generate a comparative assessment and generating a performance report based on the one or more performance metrics.

The object and advantages of the embodiments will be realized and achieved at least by the elements, features, and combinations particularly pointed out in the claims. It is to be understood that both the foregoing general description and the following detailed description are explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1 illustrates an example environment for surgical performance assessment;

FIG. 2 illustrates another example environment for surgical performance assessment;

FIG. 3 illustrates a flowchart of an example method of surgical performance assessment;

FIG. 4 is a block diagram of an example computing system for generative artificial intelligence (AI); and

FIG. 5 is a block diagram of an example computing device, all in accordance with at least some embodiments of the present disclosure.

### DETAILED DESCRIPTION

Modern surgical procedures generate video data that may capture visual information about surgical techniques, instrument movements, and procedural workflows. However, the analysis of surgical videos has traditionally relied on manual review by experienced surgeons or proctors, which may be time-consuming, subjective, and inconsistent. As a result, surgeons may lack tools to assess their surgical performance, identify areas for improvement, and/or benchmark their techniques against expert standards or their own historical performance.

Furthermore, some video management systems may rely solely on metadata-based search capabilities to search video data. For example, the system may search video data based on date, time, patient name, or surgeon name. The systems may fail to be able to search video data based on the content within the surgical videos, such as specific procedures performed and/or identify locations of the specific procedures in the video data. As a result, surgeons may not be able to easily locate in video data instances of specific procedures. For example, a surgeon may know that a specific procedure was performed during a surgery. However, the surgeon may not be able to easily locate the specific procedure in video of the surgery.

The present disclosure relates generally to systems and methods for automated surgical video analysis and performance assessment in surgery. The disclosed systems and methods may provide AI-powered analysis of surgical procedures that assists with content-based video search, automatic surgical phase recognition, objective performance assessment, and/or actionable feedback generation. For example, a surgical management system may obtain video data capturing a cataract surgery procedure, process the video data using machine learning models to automatically identify distinct surgical phases such as incision, capsulorhexis, phacoemulsification, and intraocular lens (IOL) injection, detect and track surgical instruments throughout the procedure, calculate performance metrics based on the identified phases and detected instruments, and generate a report that may include comparisons to benchmark values and historical performance data. As such, the present disclosure may enable surgeons to receive data-driven insights into their surgical performance.

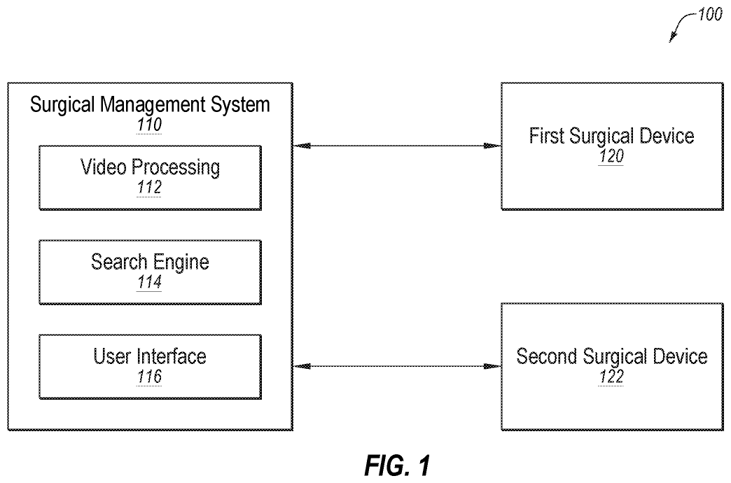

Turning to the figures, FIG. 1 illustrates an example surgical video analysis environment 100 for automated surgical performance assessment. The environment 100 may be arranged in accordance with at least one embodiment described in the present disclosure. The environment 100 may include a surgical management system 110, a first surgical device 120, and a second surgical device 122.

In some embodiments, the first surgical device 120 and the second surgical device 122 may be any devices used during surgical procedures that may be configured to capture video data. For example, the first surgical device 120 may be a surgical microscope with an integrated camera that may capture high-definition video of a surgical field during a surgery procedure. The second surgical device 122 may be another surgical device with visualization capabilities that may capture video during a surgery procedure. In some embodiments, the first surgical device 120 and the second surgical device 122 may be configured to transmit captured video data to the surgical management system 110 via wired or wireless network connections. The video data may be transmitted in real-time during the surgical procedure or may be uploaded to the surgical management system 110 after the procedure is completed.

In some embodiments, the first surgical device 120 and the second surgical device 122 may be configured to communicate bidirectionally with the surgical management system 110. For example, the first surgical device 120 and the second surgical device 122 may send video data to the surgical management system 110 and may receive control signals, configuration parameters, and/or feedback information from the surgical management system 110. As an example, the surgical management system 110 may be configured to provide real-time feedback to the first surgical device 120 and/or the second surgical device 122 during a surgical procedure based on analysis of the video data. In this example, the surgical management system 110 may detect that a surgical phase is taking longer than expected or that an instrument is being used in a suboptimal manner and may provide an alert or suggestion to the first surgical device 120 and the second surgical device 122 that may be displayed to a surgeon. For example, the surgical management system 110 may generate integrate augmented reality (AR) overlays that display suggestions and/or other information.

In some embodiments, the surgical management system 110 may be configured to receive, process, analyze, and/or manage surgical video data from one or more surgical devices, such as the first surgical device 120 and the second surgical device 122. In some embodiments, the surgical management system 110 may be any computing system or combination of computing systems configured to perform the operations described in this disclosure. For example, the surgical management system 110 may include one or more servers, cloud-based computing resources, edge computing devices, or combinations thereof. In some embodiments, the surgical management system 110 may include a video processing module 112, a search engine 114, and a user interface 116, which may be configured to perform operations as described in this disclosure, among other operations. In some embodiments, the surgical management system 110 may include computer-readable instructions that may be configured to be executed by the surgical management system 110 to perform operations described in this disclosure.

In some embodiments, the video processing module 112 may be configured to receive and process surgical video data from surgical devices such as the first surgical device 120 and the second surgical device 122. The video data may include video recordings captured during surgical procedures, such as cataract surgery, vitreoretinal surgery, glaucoma surgery, neurosurgical procedures, and orthopedic procedures among other procedures.

In some embodiments, the video processing module 112 may be configured to preprocess the video data by performing operations such as video decoding, frame extraction, image normalization, noise reduction, and region of interest extraction.

The video processing module 112 may be configured to analyze the preprocessed video data to identify surgical phases, detect and/or track surgical instruments, and calculate performance metrics.

In some embodiments, to identify the surgical phases, the video processing module 112 may be configured to utilize artificial intelligence (AI) models to perform video analysis operations. The AI models may include models such as convolutional neural networks, recurrent neural networks, temporal convolutional networks, and transformer-based architectures. For example, the video processing module 112 may employ a temporal convolutional network to segment surgical videos into phases by analyzing sequences of video frames and identifying temporal boundaries between different surgical steps. As an example, the video processing module 112 may be configured to segment a cataract surgery video into distinct phases such as incision, capsulorhexis, phacoemulsification, irrigation and aspiration, and intraocular lens injection.

In some embodiments, to detect and/or track surgical instruments, the video processing module 112 may utilize object detection models such as YOLO (You Only Look Once), Faster R-CNN (Region-based Convolutional Neural Network, or EfficientDet to detect surgical instruments. In these and other embodiments, the video processing module 112 may be configured to apply tracking algorithms such as SORT (Simple Online and Realtime Tracking) or DeepSORT to maintain object identities across frames and generate trajectories of instrument movements, such as continuous movements. For example, the video processing module 112 may be configured to detect and track surgical instruments such as phacoemulsification handpieces, forceps, and irrigation and aspiration instruments throughout the surgical procedure. In some embodiments, the video processing module 112 may utilize object detection models to detect anatomical features in individual video frames.

In some embodiments, the video processing module 112 may be configured to calculate one or more performance metrics based on the analyzed video data. For example, the video processing module 112 may calculate the performance metrics based on the surgical phases and the detected one or more surgical instruments.

In some embodiments, the video processing module 112 may be configured to calculate the performance metrics based on data generated using the surgical phases and the detected one or more surgical instruments. For example, in some embodiments, the video processing module 112 may generate phase annotation data based on the surgical phases. In these and other embodiments, the phase annotation data may include a start time and an end time for each surgical phase of the plurality of surgical phases. For example, the phase annotation data may indicate that a trocar placement phase began at 00:02:15 and ended at 00:04:30, yielding a phase duration of 2 minutes and 15 seconds for that surgical phase.

In some embodiments, the video processing module 112 may generate spatial detection data using the detected one or more surgical instruments. The spatial detection data may include bounding box coordinates for the one or more surgical instruments detected in the video data. For example, the spatial detection data may include bounding box coordinates (x1, y1, x2, y2) for a detected laparoscopic grasper in each frame, enabling calculation of instrument trajectory and workspace utilization metrics.

In some embodiments, the video processing module 112 may generate instrument motion data using the detected one or more surgical instruments. In these and other embodiments, the instrument motion data may be based on path lengths traveled by a detected surgical instrument during one of the surgical phases. For example, the video processing module 112 may calculate the path length traveled by a phacoemulsification handpiece during the phacoemulsification phase by summing Euclidean distances between consecutive instrument positions extracted from the tracking data. In these and other embodiments, the instrument motion data may include changes in acceleration values of the detected surgical instrument over time as the detected surgical instrument traverses the path length. For example, the instrument motion data may quantify smoothness of instrument movement by analyzing acceleration changes along a 15-centimeter path traveled by a needle driver during a suturing phase, where lower acceleration variance may indicate more controlled instrument handling.

In some embodiments, the video processing module 112 may generate gesture data that counts and categorizes surgical gestures performed during the surgical procedure and/or an efficiency data that assesses the motion of the surgeon and overall surgical efficiency. In these and other embodiments, the efficiency data may be determined based on detecting penalty events such as instrument drops, tissue damage, or bleeding by analyzing video frames and instrument trajectories for anomalous patterns.

In some embodiments, the video processing module 112 may use the data generated using the surgical phases and the detected one or more surgical instruments to calculate the performance metrics. In these and other embodiments, the video processing module 112 may be configured to compare the calculated performance metrics to benchmark values established by surgeons or others to generate a comparative assessment. A performance report may be generated based on the comparative assessment. The performance report may include one or more quality scores and/or identify areas for improvement.

In some embodiments, the video processing module 112 may also be configured to generate surgical flow graphs that represent the sequence of surgical steps as nodes in a directed graph with edges representing transitions between steps, where edge weights may indicate the frequency of each transition.

In some embodiments, the search engine 114 may be configured to enable content-based searching of surgical videos. The search engine 114 may be configured to generate searchable representations of video content by encoding video frames into feature vectors. To generate the searchable representations, the search engine 114 may be configured to select a subset of frames associated with each of one or more surgical phases identified in the video data. In these and other embodiments, the subset of frames may be selected based on temporal boundaries identified during the surgical phase segmentation process. For example, the video processing module 112 may identify that a phacoemulsification phase begins at timestamp 2:15 and ends at timestamp 8:42 within a surgical video. The search engine 114 may extract all frames, or a representative sampling of frames, which fall within the temporal window from 2:15 to 8:42.

In some embodiments, the search engine 114 may be configured to convert each frame of the subset of frames into a feature vector that encodes semantic meaning associated with the respective frame. In some embodiments, the conversion process may utilize a pre-trained convolutional neural network (CNN) or vision transformer model that has been trained on a large corpus of images or surgical videos. Each element of the feature vector may represent an abstract visual concept or pattern learned by the neural network during training. In these and other embodiments, the feature vector may encode semantic information such as the presence of specific surgical instruments, the appearance of anatomical structures, the spatial arrangement of objects within the frame, color distributions, texture patterns, or motion characteristics. For example, a feature vector extracted from a frame showing a phaco handpiece in contact with the lens nucleus may have high activation values in dimensions corresponding to instrument presence, metallic textures, and specific spatial configurations.

In some embodiments, the search engine 114 may be configured to aggregate the feature vectors for each of the surgical phases to generate a representation for each of the surgical phases. The aggregation may be performed using one or more mathematical operations that combine the individual feature vectors extracted from the subset of frames into a single, unified representation that captures the semantic content of the entire surgical phase.

In some embodiments, the search engine 114 may be configured to process natural language queries from users and retrieve relevant video segments based on semantic similarity between the query and the video content. For example, a surgeon may submit a query such as “find cases where phacoemulsification lasted more than 10 minutes” or “show surgeries with instrument drops during capsulorhexis,” and the search engine 114 may retrieve matching video segments based on the content of the videos.

In some embodiments, to retrieve relevant video segments based on semantic similarity, the search engine 114 may convert the natural language query into a vector. In these and other embodiments, the semantic similarity between the query and the video content may be determined based on a correlation between the vector of the natural language query and the features vectors of the video content.

In some embodiments, the user interface 116 may be configured to present surgical video data, analysis results, performance metrics, and other information to users such as surgeons, surgical trainees, administrators, or other medical staff. The user interface 116 may include graphical user interface components such as video players, timelines, charts, graphs, dashboards, and interactive elements that may enable users to navigate through surgical videos, review performance metrics, compare performance across multiple surgeries, and interact with the system through natural language queries. In some embodiments, the user interface 116 may be configured to display performance reports that may include visualizations of surgical phase durations, instrument motion metrics, comparative analyses, and recommendations for improvement.

Modifications, additions, or omissions may be made to the environment 100 without departing from the scope of the present disclosure. For example, in some embodiments, the surgical management system 110 may not include one or more of the video processing modules 112 and the search engine 114. As another example, the surgical management system 110 may communicate with additional surgical devices beyond the first surgical device 120 and the second surgical device 122. For example, the surgical management system 110 may be configured to receive video data from multiple surgical microscopes, visualization systems, or other devices within a single operating room or across multiple operating rooms in a hospital or surgical center. In these and other embodiments, the surgical management system 110 may be configured to process and analyze video data from multiple concurrent surgical procedures and may provide performance feedback and analysis for each procedure independently.

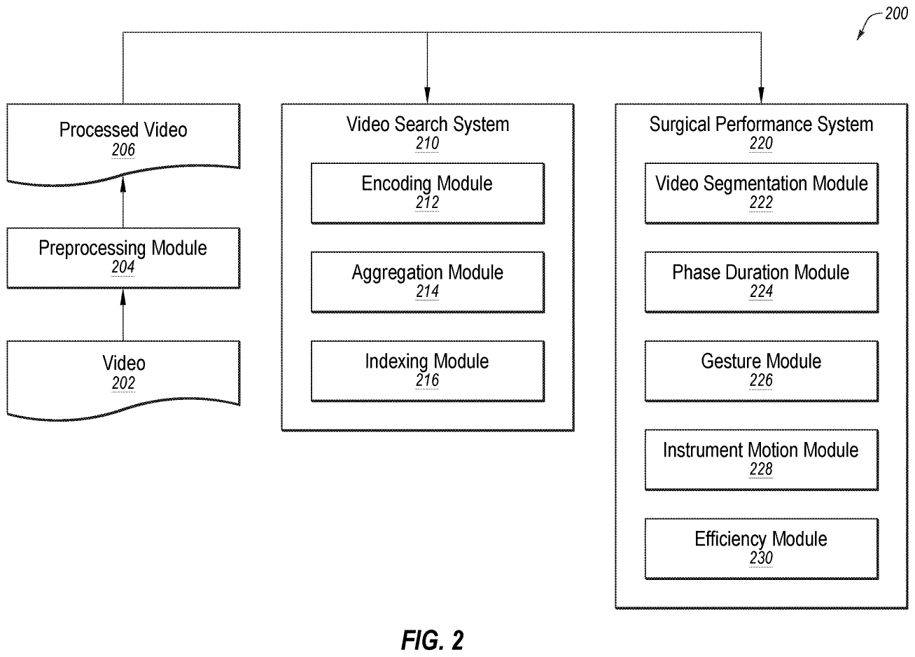

FIG. 2 illustrates an example environment 200 that may be used for surgical video analysis. The environment 200 may be arranged in accordance with at least one embodiment described in the present disclosure. The environment 200 may include a preprocessing module 204, a video search system 210, and a surgical performance system 220.

In some embodiments, any operation performed by a module in this disclosure may be implemented in software, firmware, or a combination thereof. When implemented in software, the module may comprise one or more executable instructions stored on a non-transitory computer-readable medium and configured, when executed by one or more processors, to perform the operations described herein for that module. In this manner, the functionality attributed to the module may be carried out entirely through software execution, hardware execution, or any suitable combination of the two.

In some embodiments, the preprocessing module 204 may obtain video 202. The video 202 may be surgical video data captured during a surgical procedure. The video 202 may be obtained from surgical devices such as the first surgical device 120 or the second surgical device 122 of FIG. 1. The video 202 may include video recordings of various surgical procedures. In some embodiments, the video 202 may be stored in various video formats such as MP4 (Moving Picture Experts Group (MPEG)-4), AVI (Audio Video Interleave), MOV (. MOV video file format), or other digital video formats. The video 202 may have varying resolutions, frame rates, and durations depending on the surgical procedure and recording equipment used.

In some embodiments, the preprocessing module 204 may be configured to perform preprocessing operations to prepare the video data for subsequent analysis. The preprocessing module 204 may be configured to perform operations such as video decoding to extract individual frames from the video 202, frame extraction at specified sampling rates, image normalization to standardize pixel values and color spaces, noise reduction to improve image quality, and region of interest extraction to focus on relevant portions of the surgical field. For example, the preprocessing module 204 may decode a video file encoded in H.264 format, extract frames at a rate of 30 frames per second, normalize the pixel values to a range of 0 to 1, apply a Gaussian filter to reduce noise, and crop the frames to focus on the surgical field while removing extraneous elements such as equipment interfaces or text overlays.

In some embodiments, the preprocessing module 204 may be configured to perform video stabilization to compensate for camera movement or vibrations during the surgical procedure. For example, the preprocessing module 204 may apply digital stabilization algorithms that analyze motion between consecutive frames and apply transformations to reduce unwanted movement while preserving the actual surgical actions.

In some embodiments, the preprocessing module 204 may be configured to perform color correction and brightness adjustment to account for variations in lighting conditions during the surgical procedure. Alternately or additionally, the preprocessing module 204 may analyze the color distribution and intensity levels across frames and apply corrections to ensure consistent visual appearance throughout the video. For example, the preprocessing module 204 may detect that lighting conditions changed midway through a procedure and apply a color transformation to maintain consistent appearance across all frames.

In some embodiments, the preprocessing module 204 may be configured to generate the processed video 206 as output. The processed video 206 may include the video data after preprocessing operations have been applied. The processed video 206 may have standardized format, resolution, and quality characteristics that facilitate subsequent analysis by the video search system 210 and the surgical performance system 220.

In some embodiments, the video search system 210 may be configured to enable content-based searching of surgical videos. The video search system 210 may include an encoding module 212, an aggregation module 214, and an indexing module 216. The video search system 210 may be configured to process the processed video 206 to generate searchable representations that enable users to search for surgical videos based on content rather than solely on metadata.

In some embodiments, the encoding module 212 may be configured to convert video frames from the processed video 206 into feature vectors that encode semantic information about the visual content. In some embodiments, the encoding module 212 may utilize pre-trained neural network models such as convolutional neural networks or vision transformer models to extract features from individual frames. For example, the encoding module 212 may use a ResNet-50 model pre-trained on ImageNet to extract a 512-dimensional feature vector from each frame, where each dimension of the feature vector represents activation of a learned visual pattern or concept.

In some embodiments, the encoding module 212 may be configured to process frames associated with specific surgical phases identified in the processed video 206. In some embodiments, the encoding module 212 may receive information about temporal boundaries of surgical phases from the surgical performance system 220 and may extract frames that fall within each phase. For example, if the surgical performance system 220 identifies that a phacoemulsification phase occurs from timestamp 2:15 to 8:42, the encoding module 212 may extract frames from that time interval and generate feature vectors for those frames. Alternately or additionally, the encoding module 212 may generate information about temporal boundaries of surgical phases in a similar manner to the surgical performance system 220.

In some embodiments, the encoding module 212 may be configured to sample frames at various rates depending on the characteristics of the surgical phase or the requirements of the search application. The encoding module 212 may sample frames uniformly at regular intervals, such as one frame per second, or may use adaptive sampling that selects frames based on visual content changes. For example, the encoding module 212 may sample more frames during periods of rapid motion or significant visual changes and fewer frames during static periods.

In some embodiments, the aggregation module 214 may be configured to combine the feature vectors generated by the encoding module 212 for multiple frames within a surgical phase into a single aggregated representation. The aggregation module 214 may perform mathematical operations such as averaging, max pooling, or weighted combination to generate a unified feature vector that represents the entire surgical phase. For example, the aggregation module 214 may compute the element-wise average of feature vectors from 100 frames sampled during a phacoemulsification phase to generate a single 512-dimensional feature vector representing that phase.

In some embodiments, the aggregation module 214 may be configured to use attention mechanisms to weigh the contribution of different frames when generating the aggregated representation. The aggregation module 214 may learn attention weights that indicate the relative importance of each frame for representing the surgical phase. For example, the aggregation module 214 may assign higher weights to frames that show critical moments in the surgical procedure, such as the initial contact of the phaco handpiece with the lens nucleus, and lower weights to frames showing routine portions of the procedure.

In some embodiments, the indexing module 216 may be configured to store the aggregated feature vectors generated by the aggregation module 214 in a searchable index structure. The indexing module 216 may use data structures and algorithms optimized for similarity searching in high-dimensional vector spaces, such as approximate nearest neighbor search algorithms. For example, the indexing module 216 may use a hierarchical navigable small world (HNSW) graph structure to index the feature vectors, enabling efficient retrieval of similar vectors during search operations.

In some embodiments, the indexing module 216 may be configured to associate metadata with each indexed feature vector. The metadata may include information such as the video identifier, the surgical phase identifier, the start and end timestamps of the phase, the surgeon identifier, the patient identifier, the procedure type, and other relevant information. For example, the indexing module 216 may store a feature vector along with metadata indicating that the vector represents a phacoemulsification phase from video ID 12345, occurring from timestamp 2:15 to 8:42, performed by surgeon Dr. Smith on patient 67890.

In some embodiments, the indexing module 216 may be configured to support natural language queries by converting text queries into feature vectors in the same vector space as the video feature vectors. The indexing module 216 may use vision-language models such as CLIP (Contrastive Language-Image Pre-training) that have been trained to align text and image representations in a shared embedding space. For example, when a user submits a query “show me phacoemulsification procedures lasting more than 10 minutes,” the indexing module 216 may encode the text query into a feature vector using the text encoder of a CLIP model and then search for video feature vectors that have high cosine similarity with the query vector.

In some embodiments, the indexing module 216 may be configured to combine semantic similarity search with metadata filtering to refine search results. The indexing module 216 may first retrieve candidate videos based on semantic similarity and then apply filters based on metadata criteria such as duration thresholds, surgeon identifiers, or date ranges. For example, the indexing module 216 may retrieve the top 90 videos with feature vectors most similar to a query vector and then filter to retain the videos where the phacoemulsification phase lasted more than 10 minutes. Alternately or additionally, the indexing module 216 may first retrieve candidate videos b metadata criteria such as duration thresholds, surgeon identifiers, or date ranges, and then apply semantic similarity.

In some embodiments, the surgical performance system 220 may be configured to analyze the processed video 206 to extract performance metrics related to surgical technique and efficiency. The surgical performance system 220 may include a video segmentation module 222, a phase duration module 224, a gesture module 226, an instrument motion module 228, and an efficiency module 230.

In some embodiments, the surgical performance system 220 may be configured to provide quantitative assessments of surgical performance that may be used for training, quality assurance, and improvement. Alternately or additionally, the surgical performance system 220 may be configured to provide quantitative assessments of surgical performance as feedback in real-time to assist surgeons during a surgical procedure.

In some embodiments, the video segmentation module 222, the phase duration module 224, the gesture module 226, the instrument motion module 228, and/or the efficiency module 230 may each generate one or more performance metrics that may be used to determine the quantitative assessments of a surgical performance. A discussion of how each of the video segmentation module 222, the phase duration module 224, the gesture module 226, the instrument motion module 228, and/or the efficiency module 230 may generate performance metrics follows.

In some embodiments, the video segmentation module 222 may be configured to analyze the processed video 206 to identify and delineate distinct surgical phases within the surgical procedure. The video segmentation module 222 may utilize temporal segmentation models such as temporal convolutional networks or transformer-based architectures to classify each frame or time segment of the video into one of multiple surgical phases. For example, the video segmentation module 222 may use a temporal convolutional network trained on annotated surgical videos to classify each second of a cataract surgery video into phases such as incision, capsulorhexis, hydrodissection, phacoemulsification, irrigation and aspiration, and IOL injection.

In some embodiments, the video segmentation module 222 may be configured to identify temporal boundaries between surgical phases by detecting transitions in visual features, instrument usage, or surgical actions. The video segmentation module 222 may analyze changes in frame-level features or model predictions to determine when one phase ends and another begins. For example, the video segmentation module 222 may detect that the phacoemulsification phase ends when the phaco handpiece is withdrawn from the eye and the irrigation and aspiration handpiece is introduced, marking the transition to the irrigation and aspiration phase. In some embodiments, the video segmentation module 222 may be configured to apply temporal smoothing or post-processing to refine the segmentation results and reduce spurious phase transitions. The video segmentation module 222 may use techniques such as median filtering, hidden Markov models, or conditional random fields to enforce temporal consistency in the phase predictions.

In some embodiments, the phase duration module 224 may be configured to calculate the duration of each surgical phase identified by the video segmentation module 222. The phase duration module 224 may determine the start time and end time of each phase based on the temporal boundaries identified during segmentation and compute the duration as the difference between the end time and start time. For example, the phase duration module 224 may determine that a phacoemulsification phase began at timestamp 2:15 and ended at timestamp 8:42, yielding a duration of 6 minutes and 27 seconds.

In some embodiments, the phase duration module 224 may be configured to compare the calculated phase durations to benchmark values or reference standards to assess whether the durations fall within expected ranges. In some embodiments, the phase duration module 224 may be configured to identify phases with durations that deviate significantly from expected values and flag those phases for further review or analysis.

In some embodiments, the phase duration module 224 may be configured to aggregate phase durations across multiple surgeries performed by the same surgeon to compute summary statistics such as mean duration, median duration, standard deviation, and trends over time. The phase duration module 224 may generate longitudinal analyses that show how a surgeon's phase durations have changed over their career or training period. For example, the phase duration module 224 may compute that a surgeon's mean phacoemulsification duration has decreased from 8 minutes in their first 10 cases to 5 minutes in their most recent 10 cases, indicating improvement in efficiency.

In some embodiments, the data obtained or determined by the phase duration module 224 may be used as one or more performance metrics for quantitative assessments of surgical performance.

In some embodiments, the gesture module 226 may be configured to identify and classify surgical gestures or elementary surgical actions performed during the surgical procedure. The gesture module 226 may utilize gesture recognition models that analyze short video segments or instrument trajectories to classify gestures into predefined categories such as reaching for an instrument, grasping tissue, cutting, suturing, injecting, or aspirating. For example, the gesture module 226 may use a spatiotemporal convolutional network to analyze 2-second video clips and classify each clip into one of 15 gesture categories commonly observed in cataract surgery.

In some embodiments, the gesture module 226 may be configured to count the number of occurrences of each gesture type during the surgical procedure or within specific surgical phases. The gesture module 226 may generate gesture frequency statistics that quantify how often each gesture was performed. For example, the gesture module 226 may determine that during a phacoemulsification phase, the surgeon performed 23 instances of the “grasp and rotate” gesture and 8 instances of the “inject viscoelastic” gesture.

In some embodiments, the gesture module 226 may be configured to calculate the duration of each gesture occurrence and compute summary statistics such as mean gesture duration, total time spent in each gesture type, and gesture efficiency metrics. The gesture module 226 may identify gestures that took longer than expected or were repeated more frequently than necessary.

In some embodiments, the gesture module 226 may be configured to analyze gesture sequences and transitions to identify patterns in surgical workflow. In these and other embodiments, the gesture module 226 may construct gesture transition graphs that show the frequency of transitions from one gesture type to another.

In some embodiments, the data obtained or determined by the gesture module 226 may be used as one or more performance metrics for quantitative assessments of surgical performance.

In some embodiments, the instrument motion module 228 may be configured to analyze the movement of surgical instruments throughout the surgical procedure. In these and other embodiments, the instrument motion module 228 may utilize object detection and tracking algorithms to identify surgical instruments in video frames and maintain consistent identities for each instrument across frames. After identifying the objects, the instrument motion module 228 may be configured to extract the spatial coordinates of each detected instrument in each frame. The instrument motion module 228 may store the coordinates as time-series data that represents the trajectory of the instrument over time.

For example, the instrument motion module 228 may extract the (x, y) pixel coordinates of the phaco handpiece tip in each frame at 30 frames per second, generating a trajectory consisting of 11,610 coordinate pairs for a 6-minute and 27-second phacoemulsification phase.

In some embodiments, the instrument motion module 228 may be configured to calculate path length data by computing the cumulative distance traveled by an instrument during a surgical phase or the entire procedure. The instrument motion module 228 may calculate distances, such as Euclidean distances, between consecutive instrument positions and sum those distances to obtain the total path length. For example, the instrument motion module 228 may calculate that the phaco handpiece traveled a total path length of 2.3 meters during the phacoemulsification phase by summing the distances between consecutive positions: pathlength =Σ sqrt((x{i+1}−xi)\hat{ }2+(y{i+1}−yi)\hat{ }2) for all consecutive frame pairs.

In some embodiments, the instrument motion module 228 may be configured to calculate velocity data by computing the rate of change of instrument position over time. The instrument motion module 228 may calculate instantaneous velocity for each interval and compute summary statistics such as mean velocity, maximum velocity, and velocity variance. In some embodiments, the instrument motion module 228 may be configured to calculate acceleration metrics by computing the rate of change of velocity over time. For example, the instrument motion module 228 may calculate instantaneous acceleration for each time interval and compute summary statistics such as mean acceleration, maximum acceleration, and acceleration variance.

In some embodiments, the instrument motion module 228 may be configured to calculate jerk data by computing the rate of change of acceleration over time. Jerk may be used as a measure of movement smoothness, where lower jerk values indicate smoother, more controlled movements. The instrument motion module 228 may calculate instantaneous jerk for each time interval and compute summary statistics such as mean jerk, root mean square jerk, and jerk variance.

In some embodiments, the instrument motion module 228 may be configured to calculate economy of motion data that quantify the efficiency of instrument movements. The instrument motion module 228 may compare the actual path length traveled by an instrument to an ideal or minimum path length that would be required to accomplish the same surgical task. For example, the instrument motion module 228 may determine that the phaco handpiece traveled 2.3 meters during the phacoemulsification phase, while an ideal path based on the surgical field dimensions and required movements would be 1.8 meters, yielding an economy of motion ratio of 1.8/2.3=0.78, indicating that 78% of the movement was task-relevant.

In some embodiments, the data obtained or determined by the instrument motion module 228 may be used as one or more performance metrics for quantitative assessments of surgical performance.

In some embodiments, the efficiency module 230 may be configured to calculate overall efficiency metrics that combine multiple performance indicators to provide a comprehensive assessment of surgical efficiency. The efficiency module 230 may integrate information from the phase duration module 224, the gesture module 226, and the instrument motion module 228 to compute composite efficiency scores. For example, the efficiency module 230 may calculate an overall efficiency score by combining normalized metrics for phase duration, gesture count, path length, and jerk using a weighted formula: efficiencyscore=0.3(1−normalizedduration)+0.2(1−normalizedgesturecount)+0.3 (1−normalizedpathlength)+0.2(1−normalizedjerk). The interpretation is that higher efficiency scores indicate that movement of them is more deliberate and less wasteful.

In some embodiments, the efficiency module 230 may be configured to detect penalty events such as instrument drops, tissue damage, bleeding, or other adverse performance events during the surgical procedure. The efficiency module 230 may utilize anomaly detection models or event detection models that analyze video frames and instrument trajectories to identify patterns associated with penalty events. For example, the efficiency module 230 may detect an instrument drop by identifying a sudden disappearance of an instrument from the video frames followed by a period where the instrument is not visible, or by detecting a rapid downward motion of an instrument followed by a period of no motion. In some embodiments, the efficiency module 230 may be configured to count the number of penalty events and categorize them by type and severity. The efficiency module 230 may assign penalty scores or deductions to each event based on predefined severity levels.

In some embodiments, the data obtained or determined by the efficiency module 230 may be used as one or more performance metrics for quantitative assessments of surgical performance.

In some embodiments, the surgical performance system 220 may be configured to generate efficiency reports that summarize the performance metrics and provide actionable recommendations for improvement. For example, the surgical performance system 220 may identify specific areas where the surgeon's performance deviated from benchmarks or best practices and suggest targeted interventions. For example, the surgical performance system 220 may generate a report stating: “Your phacoemulsification phase duration was 25% longer than the benchmark average. Consider optimizing your chopping technique to reduce emulsification time. Your instrument motion showed higher jerk values than expert benchmarks, suggesting opportunities to improve movement smoothness through deliberate practice.”

In some embodiments, the efficiency reports and/or the performance metrics may be presented via an application interface that includes, for example, an interactive timeline, a key metrics summary, comparative graphs, heat maps and snail tracks, trend analysis, actionable feedback, automated reports, pupil dynamics, real-time alerts, performance progression, benchmarking and goal setting, and feedback sessions. The interactive timeline may overlay segmented phases and surgical gestures with color coding. The key metrics summary may display individual numeric values for phase durations, instrument path length, velocity, acceleration, jerk, efficiency scores, and penalty counts. The comparative graphs may include side-by-side comparisons of a surgeon's metrics versus benchmarks (e.g., expert averages) using bar charts, line graphs, and scatter plots. The heat maps and snail tracks may include visual representations of instrument motion patterns over the surgical field. The trend analysis may be a time-series analysis showing performance improvements over successive procedures. The actionable feedback may generate textual and visual recommendations that may be based on metric thresholds (e.g., “Your dissection phase lasted 20% longer than the expert benchmark. Consider optimizing instrument handling to reduce unnecessary movements”).

In some embodiments, the efficiency reports may be generated after each procedure or training session that includes all computed metrics, visualizations, and targeted feedback.

In some embodiments, the surgical performance system 220 may track performance progression use Principal Component Analysis (PCA) or other dimensionality reduction techniques to visually display a surgeon's learning trajectory, with “arrows” indicating improvement between sessions. In some embodiments, the surgical performance system 220 may allow for benchmarking and goal setting that may allow trainees to set performance targets based on expert benchmarks and track progress toward these goals over time.

In some embodiments, the environment 200 may include advanced AI capabilities. For example, the environment 200 may implement self-supervised or semi-supervised learning to improve segmentation accuracy even with limited annotated data, and/or use transformer-based architectures for better handling of long video sequences.

In some embodiments, the environment 200 may provide multi-modal data fusion. For example, the environment 200 may combine video with other data sources (e.g., kinematic data, sensor outputs, patient vital signs) for comprehensive performance analysis, and/or use deep learning models that can simultaneously process visual, textual, and numerical data.

In some embodiments, the environment 200 may provide enhanced GUI (Graphical User Interface) interactivity. For example, the environment 200 may introduce customizable dashboards and personalized learning modules for surgeons, and/or facilitate remote collaboration and peer review through integrated telemedicine and sharing features.

In some embodiments, the environment 200 may provide predictive analytics. For example, the environment 200 may incorporate predictive models that forecast surgical outcomes based on historical performance metrics, and/or use trend analysis to recommend targeted training or technique adjustments.

In some embodiments, the environment 200 may provide scalability and integration. For example, the environment 200 may ensure seamless integration with hospital data management systems (e.g., PACS (Picture Archiving and Communication System), EMR (Electronic Medical Record)), and/or optimize the system for various surgical specialties beyond ophthalmology, such as neurosurgery and orthopedics.

Modifications, additions, or omissions may be made to the environment 200 without departing from the scope of the present disclosure. For example, in some embodiments, the video search system 210 and the surgical performance system 220 may share components or may be integrated into a single unified system. As another example, the environment 200 may include additional modules for analyzing other aspects of surgical performance, such as modules for analyzing surgeon ergonomics, patient positioning, or equipment utilization. In these and other embodiments, the environment 200 may be configured to process video data from multiple surgical procedures simultaneously and may provide comparative analyses across different procedures, surgeons, or institutions.

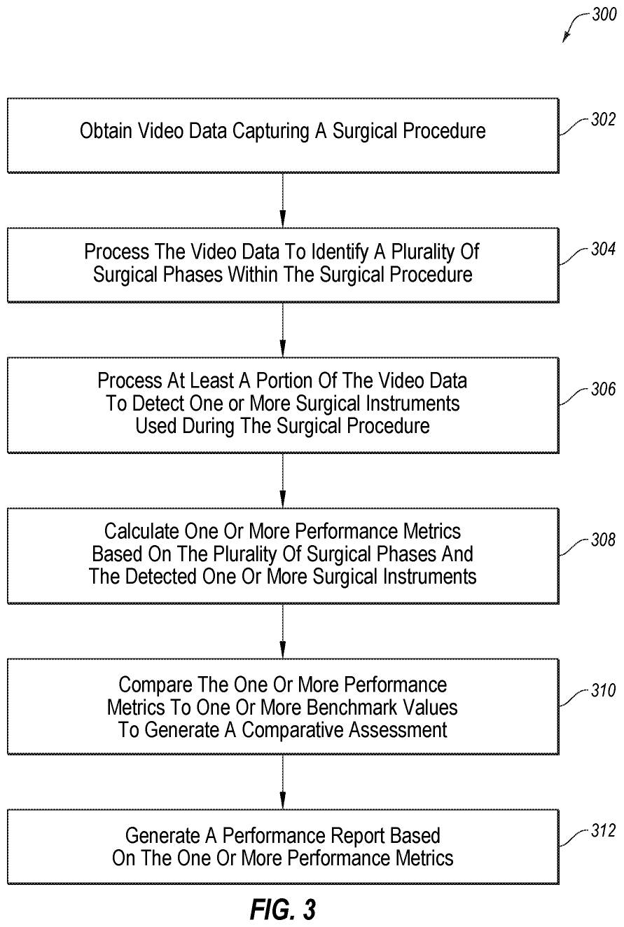

FIG. 3 illustrates a flowchart of an example method 300 for assessing a surgical procedure from video data. The method 300 may be arranged in accordance with at least one embodiment described in the present disclosure. One or more operations of the method 300 may be performed, in some embodiments, by a device or system, such as the surgical management system 110 of FIG. 1, the surgical performance system 220 of FIG. 2, or another device or combination of devices. In these and other embodiments, the method 300 may be performed based on the execution of instructions stored on one or more non-transitory computer-readable media. Although illustrated as discrete blocks, various blocks may be divided into additional blocks, combined into fewer blocks, or eliminated, depending on the desired implementation.

The method 300 may begin at block 302, where video data capturing a surgical procedure may be obtained. The video data may be obtained from surgical devices.

At block 304, the video data may be processed to identify surgical phases within the surgical procedure. The processing may involve analyzing sequences of video frames using AI models such as temporal convolutional networks or transformer-based architectures. The models may classify each frame or time segment of the video into one of multiple predefined surgical phases.

At block 306, at least a portion of the video data may be processed to detect one or more surgical instruments used during the surgical procedure.

At block 308, one or more performance metrics may be calculated based on the plurality of surgical phases and the detected one or more surgical instruments. The performance metrics may include phase duration metrics, instrument motion metrics, gesture metrics, and efficiency metrics. The performance metrics may provide quantitative assessments of surgical technique and efficiency. For example, performance metrics may include the duration of each surgical phase, the path length traveled by each instrument, the velocity and acceleration of instrument movements, the jerk values indicating movement smoothness, the number and types of surgical gestures performed, and the number of penalty events such as instrument drops or tissue damage.

In some embodiments, calculating performance metrics may include generating phase annotation data including a start time and an end time for each surgical phase. The phase annotation data may be used to calculate phase durations by computing the difference between the end time and start time for each phase.

In some embodiments, calculating performance metrics may include generating spatial detection data including bounding box coordinates for the detected surgical instruments. The spatial detection data may be used to extract instrument positions and calculate motion metrics.

In some embodiments, calculating performance metrics may include generating instrument motion data based on path lengths traveled by a detected surgical instrument during one of the surgical phases. In some embodiments, calculating performance metrics may include determining changes in acceleration values of the detected surgical instrument over time as the detected surgical instrument traverses the path length.

In some embodiments, calculating performance metrics may include calculating jerk values by computing the rate of change of acceleration over time. Jerk may be used as a measure of movement smoothness, where lower jerk values indicate smoother, more controlled movements. In some embodiments, the one or more performance metrics may include a gesture metric that counts and categorizes surgical gestures performed during the surgical procedure. In some embodiments, the one or more performance metrics may include an efficiency metric that assesses the motion of the surgeon and overall surgical efficiency.

At block 310, the one or more performance metrics may be compared to one or more benchmark values to generate a comparative assessment. The benchmark values may be established by expert surgeons, derived from statistical distributions of historical surgical data, or defined based on clinical guidelines or best practices.

At block 312, a performance report may be generated based on the one or more performance metrics. The performance report may include visualizations of the performance metrics, the comparative assessment, and recommendations for improvement. The performance report may be presented to the surgeon or other users through a user interface. For example, the performance report may include a timeline visualization showing the duration of each surgical phase, charts comparing the surgeon's metrics to benchmark values, graphs showing instrument motion trajectories, and text recommendations.

In some embodiments, generating the performance report may include calculating one or more quality scores based on the performance metrics and the comparative assessment. The quality scores may provide an overall assessment of surgical quality or may assess specific aspects of surgical technique. For example, an overall quality score may be calculated by combining normalized metrics for phase duration, instrument motion, gesture efficiency, and penalty events using a weighted formula, and the quality score may be presented on a scale of 0 to 100, where higher scores indicate better performance.

It is understood that, for the method 300 and other processes and methods disclosed herein, the functions and/or operations performed may be implemented in differing orders. Furthermore, the outlined functions and operations are only provided as examples, and some of the functions and operations may be optional, combined into fewer functions and operations, or expanded into additional functions and operations without detracting from the essence of the disclosed embodiments.

For example, the method 300 may further include providing real-time feedback to a surgeon performing the surgical procedure based on the performance report.

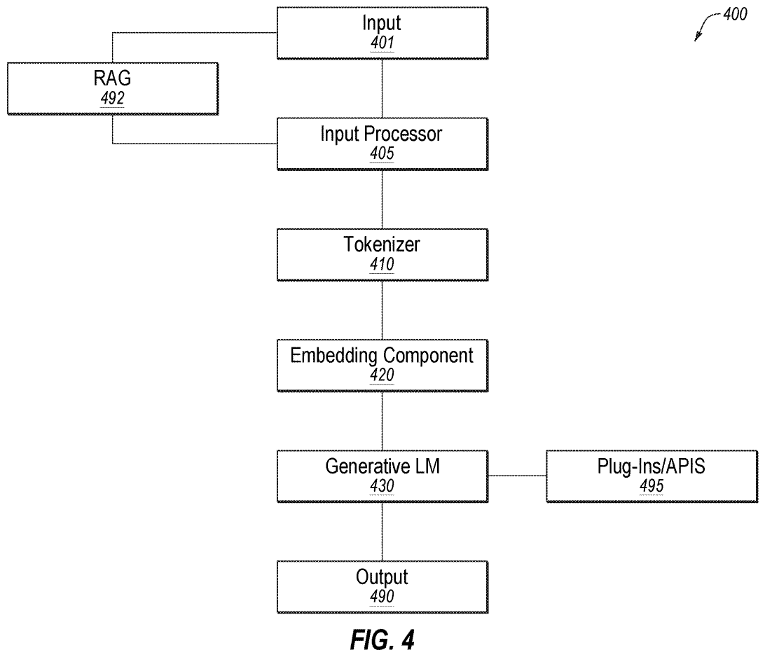

FIG. 4 is a block diagram of an example generative language model system 400 suitable for use in implementing at least some embodiments of the present disclosure. In the example illustrated in FIG. 4, the generative language model system 400 includes a retrieval augmented generation (RAG) component 492, an input processor 405, a tokenizer 410, an embedding component 420, plug-ins/APIs 495, and a generative language model (LM) 430 (which may include an LLM (large language model), a VLM (vision language model), a MMLM (multi-modal language model), a VLA (vision language action) model, etc.). The generative language model system 400 may be an example of a system that performs the AI models in the modules described with respect to FIG. 2.

At a high level, the input processor 405 may receive an input 401 comprising text and/or other types of input data (e.g., audio data, video data, frame data, sensor data), depending on the architecture of the generative LM 430 (e.g., LLM/LRM (Large Reasoning Model)/VLM/MMLM/etc.). Taking raw input text as an example, the input processor 405 may prepare raw input text in various ways. For example, the input processor 405 may perform various types of text filtering to remove noise (e.g., special characters, punctuation, HTML (Hypertext Markup Language) tags, stopwords, portions of a frame(s), portions of audio, etc.) from relevant textual content.

In some embodiments, a RAG component 492 (which may include one or more RAG models, and/or may be performed using the generative LM 430 itself) may be used to retrieve additional information to be used as part of the input 401 or prompt. RAG may be used to enhance the input to the LLM/LRM/VLM/MMLM/etc. with external knowledge, so that answers to specific questions or queries or requests are more relevant-such as in a case where specific knowledge is required. The RAG component 492 may fetch this additional information (e.g., grounding information, such as grounding text/frame/video/audio/USD/CAD/etc.) from one or more external sources, which can then be fed to the LLM/LRM/VLM/MMLM/etc. along with the prompt to improve accuracy of the responses or outputs of the model.

For example, in some embodiments, the input 401 may be generated using the query or input to the model (e.g., a question, a request, etc.) in addition to data retrieved using the RAG component 492. In some embodiments, the input processor 405 may analyze the input 401 and communicate with the RAG component 492 (or the RAG component 492 may be part of the input processor 405, in embodiments) in order to identify relevant text and/or other data to provide to the generative LM 430. The RAG component 492 may use various RAG techniques such as, naïve RAG, modular RAG techniques, and/or Graph RAG.

The tokenizer 410 may segment the (e.g., processed) text data into smaller units (tokens) for subsequent analysis and processing. The tokens may represent individual words, subwords, characters, portions of audio/video/frame/etc., depending on the implementation. For example, the tokenizer 410 may convert the (e.g., processed) text into a structured format according to tokenization schema being implemented in the particular embodiment.

The embedding component 420 may use any known embedding technique to transform discrete tokens into (e.g., dense, continuous vector) representations of semantic meaning. For example, the embedding component 420 may use pre-trained word embeddings (e.g., Word2Vec, GloVe, or FastText), one-hot encoding, Term Frequency-Inverse Document Frequency (TF-IDF) encoding, one or more embedding layers of a neural network, and/or otherwise. In some implementations in which the input 401 includes video data, the input processor 401 may extract frames or apply resizing to extracted frames, and the embedding component 420 may extract features such as optical flow embeddings or video embeddings and/or may encode temporal information or sequences of frames.

The generative LM 430 and/or other components of the generative LM system 400 may use different types of neural network architectures depending on the implementation. For example, transformer-based architectures such as those used in models like GPT (Generative Pre-trained Transformer) may be implemented and may include self-attention mechanisms. Some other non-limiting example architectures include transformers (e.g., encoder-decoder, decoder only, multi-modal), RNNs (Recurrent Neural Network), LSTMs (Long Short-Term Memory), fusion models, diffusion models, cross-modal embedding models that learn joint embedding spaces, graph neural networks (GNNs), hybrid architectures combining different types of architectures adversarial networks like generative adversarial networks or GANs or adversarial autoencoders (AAEs) for joint distribution learning, linear-time sequence modeling with selective state space modeling (SSM) architectures (e.g., Mamba LLM architectures), and/or others. As such, depending on the implementation and architecture, the embedding component 420 may apply an encoded representation of the input 401 to the generative LM 430, and the generative LM 430 may process the encoded representation of the input 401 to generate an output 490, which may include responsive text and/or other types of data.

As described herein, in some embodiments, the generative LM 430 may be configured to access or use—or capable of accessing or using—plug-ins/APIs 495 (which may include one or more plug-ins, application programming interfaces (APIs), databases, data stores, repositories, etc.). These and other architectures for LLMs/VLMs/MMLMs/VLAs/etc. described herein are meant simply as examples, and other suitable architectures may be implemented within the scope of the present disclosure.

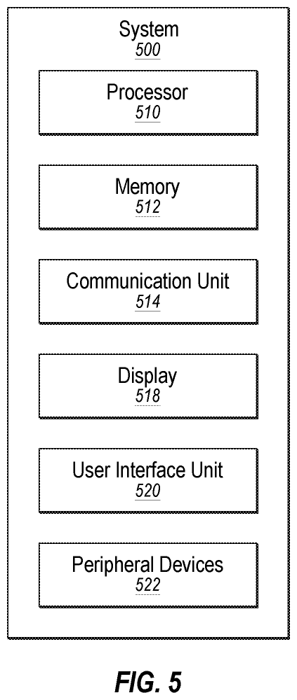

FIG. 5 illustrates an example system 500 that may be used during transcription of communications. The system 500 may be arranged in accordance with at least one embodiment described in the present disclosure. The system 500 may include a processor 510, memory 512, a communication unit 516, a display 518, a user interface unit 520, and a peripheral device 522, which all may be communicatively coupled. In some embodiments, the system 500 may be part of any of the systems or devices described in this disclosure. For example, the system 500 may be part of any of the systems or devices described in this disclosure.

Generally, the processor 510 may include any suitable special-purpose or general-purpose computer, computing entity, or processing device including various computer hardware or software modules and may be configured to execute instructions stored on any applicable computer-readable storage media. For example, the processor 510 may include a microprocessor, a microcontroller, a parallel processor such as a graphics processing unit (GPU) or tensor processing unit (TPU), a digital signal processor (DSP), an application-specific integrated circuit (ASIC), a Field-Programmable Gate Array (FPGA), quantum computer, or any other digital or analog circuitry configured to interpret and/or to execute program instructions and/or to process data.

Although illustrated as a single processor in FIG. 5, it is understood that the processor 510 may include any number of processors distributed across any number of networks or physical locations that are configured to perform individually or collectively any number of operations described herein. In some embodiments, the processor 510 may interpret and/or execute program instructions and/or process data stored in the memory 512. In some embodiments, the processor 510 may execute the program instructions stored in the memory 512.

For example, in some embodiments, the processor 510 may execute program instructions stored in the memory 512 that are related to selecting a transcription generation technique for a communication session such that the system 500 may perform or direct the performance of the operations associated therewith as directed by the instructions. In these and other embodiments, the instructions may be used to perform one or more operations of the methods described in this disclosure.

The memory 512 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 510.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store particular program code in the form of computer-executable instructions or data structures and which may be accessed by a general-purpose or special-purpose computer. Combinations of the above may also be included within the scope of computer-readable storage media.

Computer-executable instructions may include, for example, instructions and data configured to cause the processor 510 to perform a certain operation or group of operations as described in this disclosure. In these and other embodiments, the term “non-transitory” as explained in the present disclosure should be construed to exclude only those types of transitory media that were found to fall outside the scope of patentable subject matter in the Federal Circuit decision of In re Nuijten, 500 F.4d 1446 (Fed. Cir. 2007). Combinations of the above may also be included within the scope of computer-readable media.

The communication unit 516 may include any component, device, system, or combination thereof that is configured to transmit or receive information over a network. In some embodiments, the communication unit 516 may communicate with other devices at other locations, the same location, or even other components within the same system. For example, the communication unit 516 may include a modem, a network card (wireless or wired), an infrared communication device, a wireless communication device (such as an antenna), and/or chipset (such as a Bluetooth device, an IEEE 802.6 device (e.g., Metropolitan Area Network (MAN)), a WiFi device, a WiMax device, cellular communication facilities, etc.), and/or the like. The communication unit 516 may permit data to be exchanged with a network and/or any other devices or systems described in the present disclosure.

The display 518 may be configured as one or more displays, like an LCD (liquid crystal display), LED (light emitting diode), OLED (organic light emitting diode), Braille terminal, or other type of display. The display 518 may be configured to present video, text captions, user interfaces, and other data as directed by the processor 510. The user interface unit 520 may include any device to allow a user to interface with the system 500. For example, the user interface unit 520 may include a mouse, a track pad, a keyboard, buttons, camera, joysticks, foot pedals, and/or a touchscreen, among other devices. The user interface unit 520 may receive input from a user and provide the input to the processor 510. In some embodiments, the user interface unit 520 and the display 518 may be combined.

The peripheral devices 522 may include one or more devices. For example, the peripheral devices may include a microphone, a framer, and/or a speaker, among other peripheral devices. Modifications, additions, or omissions may be made to the system 500 without departing from the scope of the present disclosure. For example, in some embodiments, the system 500 may include any number of other components that may not be explicitly illustrated or described. Further, depending on certain implementations, the system 500 may not include one or more of the components illustrated and described.

As indicated above, the embodiments described herein may include the use of a special purpose or general-purpose computer (e.g., the processor 510 of FIG. 5) including various computer hardware or software modules, as discussed in greater detail below. Further, as indicated above, embodiments described herein may be implemented using computer-readable media (e.g., the memory 512 of FIG. 5) for carrying or having computer-executable instructions or data structures stored thereon.

Generally speaking, network environments suitable for use in implementing embodiments of the disclosure may include one or more client devices, servers, network attached storage (NAS), other backend devices, and/or other device types. The client devices, servers, and/or other device types (e.g., each device) may be implemented on one or more instances of the computing systems(s) 500 of FIG. 5—e.g., each device may include similar components, features, and/or functionality of the computing systems(s) 500. In addition, where backend devices (e.g., servers, NAS, etc.) are implemented, the backend devices may be included as part of a data center (such as, but not limited to, those described herein).

Components of a network environment may communicate with each other via a network(s), which may be wired, wireless, or both. The network may include multiple networks, or a network of networks. Compatible network environments may include one or more peer-to-peer network environments—in which case a server may not be included in a network environment—and one or more client-server network environments—in which case one or more servers may be included in a network environment. In peer-to-peer network environments, functionality described herein with respect to a server(s) may be implemented on any number of client devices.

In at least one embodiment, a network environment may include one or more cloud-based network environments, a distributed computing environment, a combination thereof, etc. A cloud-based network environment may be private (e.g., limited to a single organization), may be public (e.g., available to many organizations), and/or a combination thereof (e.g., a hybrid cloud environment).

The various features illustrated in the drawings may be, but are not necessarily, drawn to scale. The illustrations presented in the present disclosure are not meant to be actual views of any particular apparatus (e.g., device, system, etc.) or method but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used in the present disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” among others).

Relative terms used in the present disclosure and especially in the appended claims (e.g., bodies of the appended claims) are generally intended as falling within manufacturing tolerances and/or within scope reasonably understood by a person of skill in the art. For example, if two components are identified as being the “same” size, there may be variations consistent with manufacturing variances. Terms describing “approximately,” “similar,” “substantially,” or other terms designating similarity may convey within ten percent of the comparative value. For example, two components that are approximately the same size would be understood to be of a size within ten percent of each other.

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absent a showing that the terms “first,” “second,” “third,” etc., connote a specific order, these terms should not be understood to connote a specific order. Furthermore, absent a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited in the present disclosure are intended for pedagogical objects to aid the reader in understanding the invention and the concepts contributed by the inventor to furthering the art and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. A method of assessing a surgical procedure from a video, the method comprising:
obtaining video data capturing a surgical procedure;
processing the video data to identify a plurality of surgical phases within the surgical procedure;
processing at least a portion of the video data to detect one or more surgical instruments used during the surgical procedure;
calculating one or more performance metrics based on the plurality of surgical phases and the detected one or more surgical instruments;
comparing the one or more performance metrics to one or more benchmark values to generate a comparative assessment; and
generating a performance report based on the one or more performance metrics.

2. The method of claim 1, wherein the video data is obtained in real-time during the surgical procedure, the method further comprising providing real-time feedback to a surgeon performing the surgical procedure based on the performance report.

3. The method of claim 1, further comprising generating phase annotation data including a start time and an end time for each surgical phase of the plurality of surgical phases, wherein the one or more performance metrics are calculated based on the phase annotation data.

4. The method of claim 1, further comprising generating spatial detection data including bounding box coordinates for the one or more surgical instruments detected in the video data, wherein the one or more performance metrics are calculated based on the spatial detection data.

5. The method of claim 1, further comprising generating instrument motion data based on path lengths traveled by a detected surgical instrument during one of the surgical phases and changes in acceleration values of the detected surgical instrument over time as the detected surgical instrument traverses the path length, wherein the one or more performance metrics are calculated based on the instrument motion data.

6. The method of claim 1, further comprising:
processing at least a portion of the video data to detect one or more surgical gestures performed during the surgical procedure; and
detecting one or more adverse performance events based on one or more surgical gestures, wherein the one or more performance metrics are calculated based on the adverse performance events.

7. The method of claim 1, further comprising:
selecting a subset of frames associated with one of the plurality of surgical phases;
converting each frame of the subset of frames into a feature vector that encodes semantic meaning associated with the respective frame; and
aggregating the feature vectors to generate a representation of the one of the plurality of surgical phases.

8. The method of claim 7, further comprising:
obtaining a text query;
converting the text query into a vector; and
selecting the one of the plurality of surgical phases as being associated with the text query in response to a correlation between the vector and the aggregated feature vectors.

9. A system, the system comprising:
one or more computer-readable media configured to store instructions; and
one or more processors coupled to the one or more computer-readable media and configured to execute the instructions to cause the system to perform operations, the operations comprising:
obtaining video data capturing a surgical procedure;
processing the video data to identify a plurality of surgical phases within the surgical procedure;
processing at least a portion of the video data to detect one or more surgical instruments used during the surgical procedure;
calculating one or more performance metrics based on the plurality of surgical phases and the detected one or more surgical instruments; and
comparing the one or more performance metrics to one or more benchmark values to generate a comparative assessment; and
generating a performance report based on the one or more performance metrics.

10. The system of claim 9, wherein the video data is obtained in real-time during the surgical procedure, wherein the operations further comprise providing real-time feedback to a surgeon performing the surgical procedure based on the performance report.

11. The system of claim 9, wherein the operations further comprise generating phase annotation data including a start time and an end time for each surgical phase of the plurality of surgical phases, wherein the one or more performance metrics are calculated based on the phase annotation data.

12. The system of claim 9, wherein the operations further comprise generating spatial detection data including bounding box coordinates for the one or more surgical instruments detected in the video data, wherein the one or more performance metrics are calculated based on the spatial detection data.

13. The system of claim 9, wherein the operations further comprise generating instrument motion data based on path lengths traveled by a detected surgical instrument during one of the surgical phases and changes in acceleration values of the detected surgical instrument over time as the detected surgical instrument traverses the path length, wherein the one or more performance metrics are calculated based on the instrument motion data.

14. The system of claim 9, wherein the operations further comprise:
selecting a subset of frames associated with one of the plurality of surgical phases;
converting each frame of the subset of frames into a feature vector that encodes semantic meaning associated with the respective frame; and
aggregating the feature vectors to generate a representation of the one of the plurality of surgical phases.

15. The system of claim 14, wherein the operations further comprise:
obtaining a text query;
converting the text query into a vector; and
selecting the one of the plurality of surgical phases as being associated with the text query in response to a correlation between the vector and the aggregated feature vectors.
